(19) United States
(12) Patent Application Publication
(10) Pub. No.: US 2021/0030346 A1
(43) Pub. Date: Feb. 4, 2021

(54) BLOOD SAMPLE GUIDING INSTRUMENT AND BLOOD TEST KIT

(71) Applicant: FUJIFILM Corporation, Tokyo (JP)

(72) Inventors: Osamu NOGUCHI, Ashigarakami-gun (JP); Shinya SUGIMOTO, Tokyo (JP)

(73) Assignee: FUJIFILM Corporation, Tokyo (JP)

(21) Appl. No.: 17/076,228

(22) Filed: Oct. 21, 2020

Related U.S. Application Data
(63) Continuation of application No. PCT/JP2019/018016, filed on Apr. 26, 2019.

(30) Foreign Application Priority Data
May 15, 2018 (JP) 2018-093814

(51) Int. Cl. A61B 5/15; A61B 5/151

(52) U.S. Cl. CPC A61B 5/150748; A61B 5/15101; A61B 5/150755; A61B 5/150068

(57) ABSTRACT

Provided are a blood sample guiding instrument and a blood test kit which enable blood ejected from a finger to be guided to a storing instrument. A blood sample guiding instrument used in a blood test kit includes a cylindrical body in which a first opening and a second opening communicating with the first opening are defined and which comes into contact with a finger; and a clamping portion that is attached to an outer circumferential surface of the cylindrical body, clamps a finger, and presses the cylindrical body against the finger.

### CROSS-REFERENCE TO RELATED APPLICATIONS

The present application is a Continuation of PCT International Application No. PCT/JP2019/018016 filed on Apr. 26, 2019 claiming priority under 35 U.S.C § 119(a) to Japanese Patent Application No. 2018-093814 filed on May 15, 2018. Each of the above applications is hereby expressly incorporated by reference, in its entirety, into the present application.

### BACKGROUND OF THE INVENTION

### 1. Field of the Invention

The present invention relates to a blood sample guiding instrument and a blood test kit.

### 2. Description of the Related Art

As blood collection, in general, there are general blood collection in which a qualified person such as a doctor collects blood from the vein using a syringe, and self-blood collection in which a subject to be tested pricks his finger and the like using a blood collection needle so as to collect blood.

The blood collected by the general blood collection is transported to a medical institution or a test institution in a state of being sealed in a blood collection container, and tests are performed therein. In a case where the blood sample is transported without separating blood cells and blood plasma, tests are performed after a medical institution or a test institution separates the blood sample into blood cells and blood plasma with a centrifuge. In addition, in the self-blood collection which is performed by a subject to be tested, the collected blood sample is separated into blood cells and blood plasma by a separation membrane, the blood is transported to a test lab in this separated state, and then tests are performed therein.

In order to self-collect a blood sample, a blood sample guiding instrument is used in many cases. For example, JP2010-502278A discloses an integrated device including a skin piercing member, and a pressure member configured to apply pressure to a collection site.

### SUMMARY OF THE INVENTION

Meanwhile, after puncturing a finger with the skin piercing member, it is necessary to transfer the blood ejected from the finger to a storing instrument. However, according to JP2010-502278A, there is a concern that the blood that has been ejected from the finger cannot be reliably transferred to the storing instrument after being separated from the finger.

The present invention has been made in view of such circumstances, and an object of the present invention is to provide a blood sample guiding instrument and a blood test kit which enable blood ejected from a finger to be guided to a storing instrument.

A blood sample guiding instrument according to a first aspect is a blood sample guiding instrument used in a blood test kit and comprises a cylindrical body in which a first opening and a second opening communicating with the first opening are defined and which comes into contact with a finger; and a clamping portion that is attached to an outer circumferential surface of the cylindrical body, clamps a finger, and presses the cylindrical body against the finger.

In the blood sample guiding instrument according to a second aspect, a shape of a part of the cylindrical body which comes into contact with the finger is a curved shape protruding toward a finger side in a top view.

In the blood sample guiding instrument according to a third aspect, the first opening of the cylindrical body is larger than the second opening, and at least a part of an inner circumferential surface of the cylindrical body forms a tapered surface.

In the blood sample guiding instrument according to a fourth aspect, the clamping portion includes a support member, and at least two binding members that are disposed to be spaced from each other.

In the blood sample guiding instrument according to a fifth aspect, the binding member adjusts a clamping force for the finger.

In the blood sample guiding instrument according to a sixth aspect, the binding member is provided at a positioning portion provided on the outer circumferential surface of the cylindrical body.

In the blood sample guiding instrument according to a seventh aspect, an inner circumferential surface of the cylindrical body has water repellency.

The blood sample guiding instrument according to an eighth aspect further comprises, on a second opening side of the cylindrical body, a connecting portion that is connected to an opening of a storing instrument storing a diluent solution.

A blood test kit according to a ninth aspect comprises the above-described blood sample guiding instrument which collects a blood sample; a diluent solution that dilutes the collected blood sample; and a storing instrument that stores the diluted blood sample, in which a concentration of a target component in the blood sample is analyzed using a standard component homeostatically present in blood or a standard component that is not present in blood but is contained in the diluent solution.

The blood test kit according to a tenth aspect, further comprises a separating instrument that separates and recovers blood plasma from the diluted blood sample.

With the blood sample guiding instrument and the blood test kit according to the aspects of the present invention, blood ejected from a finger can be guided to a storing instrument.

### BRIEF DESCRIPTION OF THE DRAWINGS

FIG. 1 is a perspective view showing an example of a blood sample guiding instrument.

FIG. 2 is a perspective view of FIG. 1 viewed from another direction.

FIG. 3 is a cross-sectional view of the blood sample guiding instrument.

FIG. 4 is a top view of the blood sample guiding instrument.

FIG. 5 is a view showing an example of a configuration of a storing instrument that stores a diluted blood sample.

FIG. 6 is an explanatory view illustrating a method of using the blood sample guiding instrument.

FIG. 7 is an explanatory view illustrating a method of using the blood sample guiding instrument.

FIG. 8 is a view showing an example of a holding instrument that holds a separating instrument.

FIG. 9 is a cross-sectional view showing an action of the separating instrument.

FIG. 10 is a cross-sectional view showing an action of the separating instrument.

### DESCRIPTION OF THE PREFERRED EMBODIMENTS

Hereinafter, preferred embodiments of the present invention will be described with reference to attached drawings. The present invention will be explained by the following preferred embodiments. Modifications can be made by many methods without departing from the scope of the present invention and other embodiments besides the embodiments can be used. Accordingly, all modifications within the scope of the present invention are included in the scope of the claims. In the present specification, in a case where numerical ranges are expressed using “to,” the numerical range also includes numerical values of an upper limit and a lower limit indicated by “to.” A standard component which is homeostatically present in blood may be referred to as an external standard substance or an external standard. In addition, a standard component which is not present in blood may be referred to as an internal standard substance or an internal standard.

<Blood Sample Guiding Instrument>

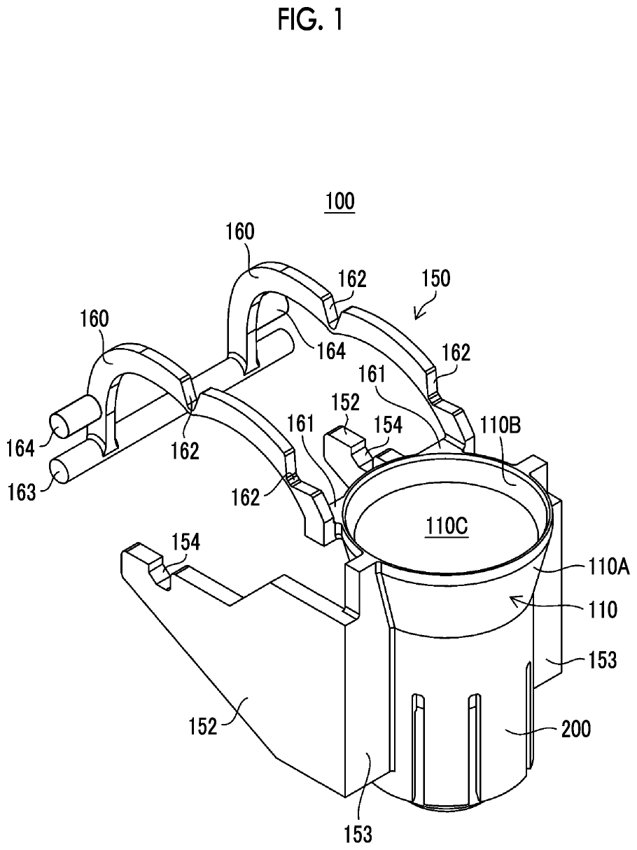

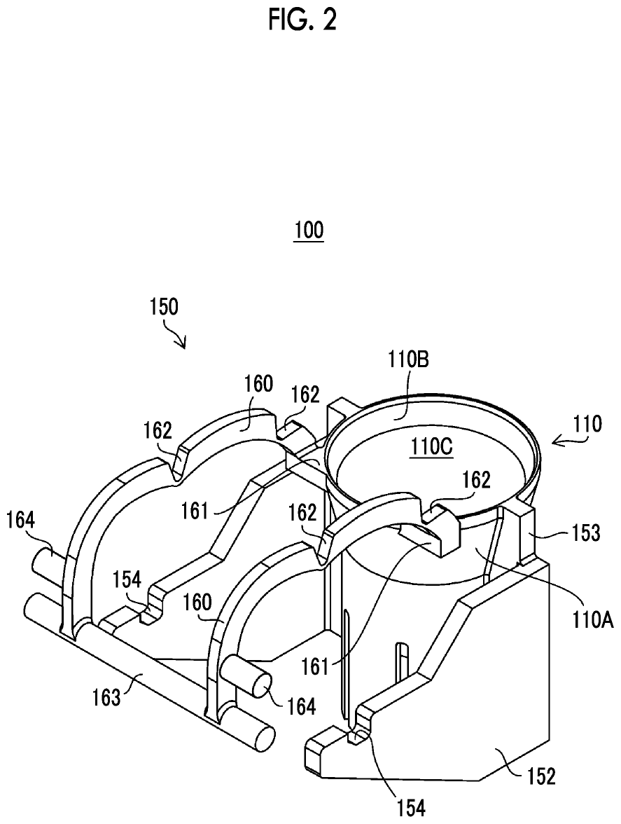

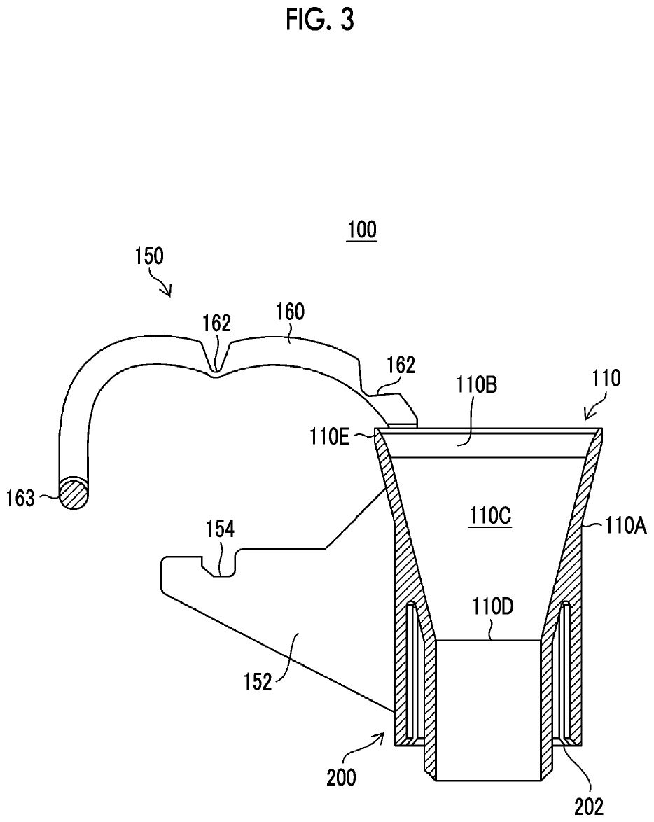

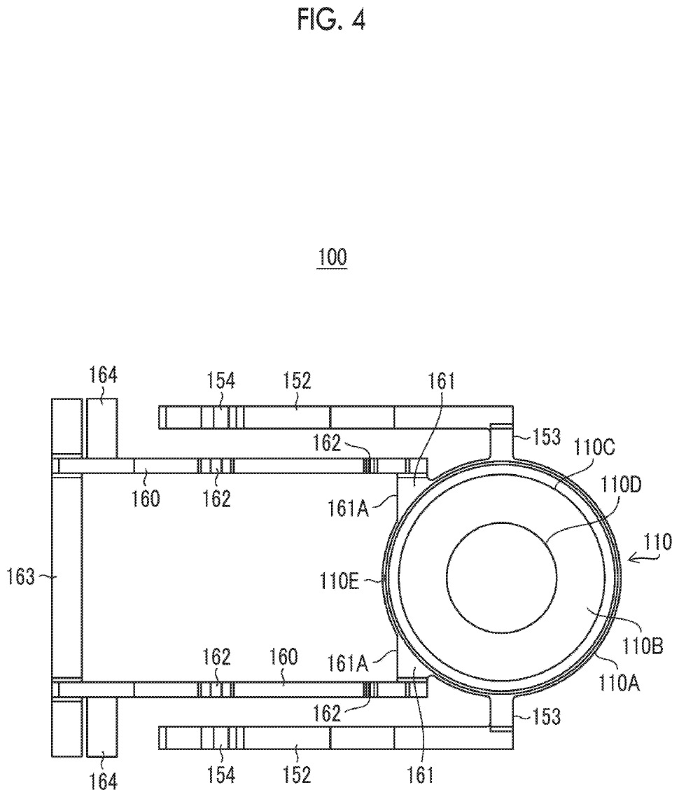

A blood sample guiding instrument of the embodiment will be described based on FIGS. 1 to 4. FIGS. 1 and 2 are perspective views of the blood sample guiding instrument, FIG. 3 is a cross-sectional view of the blood sample guiding instrument, and FIG. 4 is a top view of the blood sample guiding instrument.

A blood sample guiding instrument 100 includes a cylindrical body 110 that comes into contact with a finger, and a clamping portion 150 attached to an outer circumferential surface 110A of the cylindrical body 110. The clamping portion 150 clamps a finger and presses the cylindrical body against the finger. By clamping the finger, the blood pressure of the finger in the clamped region can be increased. By puncturing a region with high blood pressure using an instrument attached with a knife such as a lancet, blood can be easily ejected from a finger. By pressing the cylindrical body 110 against a region from which blood is ejected, the blood can be separated from the finger, and the blood can be transferred to the cylindrical body 110. Through the blood sample guiding instrument 100, the blood can be guided to a storing instrument for a test and analysis, and the blood can be transferred. In a case where blood is subjected to a test and analysis, it is called a blood sample.

As shown in FIG. 3, a first opening 110C and a second opening 110D are defined in the cylindrical body 110 of the blood sample guiding instrument 100, and the cylindrical body 110 has a hollow structure in which the first opening 110C and the second opening 110D communicate with each other. In the embodiment, an opening area of the first opening 110C is larger than an opening area of the second opening 110D. An inner circumferential surface 110B of the cylindrical body 110 has a tapered surface that widens from the second opening 110D toward the first opening 110C. By forming the inner circumferential surface 110B into a tapered surface, a blood sample can be easily dropped from the first opening 110C toward the second opening 110D.

The clamping portion 150 is composed of two support members 152 that support fingers, and at least two binding members 160 that are disposed to be spaced from each other. The two support members 152 are spaced from each other and are disposed at opposing positions. As shown in FIG. 1, the support member 152 is disposed on the outer circumferential surface 110A of the cylindrical body 110 via a connecting portion 153. A distance between the two support members 152 is larger than a distance of an outer diameter of the cylindrical body 110. In the support member 152, a cutout portion 154 is formed at a position spaced from the cylindrical body 110.

The two binding members 160 are provided on the cylindrical body 110 via a positioning portion 161 provided on the outer circumferential surface 110A on the first opening 110C side. As shown in FIG. 4, two positioning portions 161 each have a flat surface 161A that comes into contact with a finger. The outer circumferential surface 110A of the cylindrical body 110, which is located between the two positioning portions 161, serves as a contact part 110E that comes into contact with a finger. A part of the outer circumferential surface 110A constitutes the contact part 110E. As shown in FIG. 4, a shape of the cylindrical body 110 in the contact part 110E has a curved shape that protrudes toward the finger side in a top view. In a case where the contact part has a curved shape protruding toward the finger side, the outer circumferential surface 110A of the cylindrical body 110 can be pressed deeply against the finger, and thereby blood can be easily separated from the finger. As for a shape of the cylindrical body 110, the contact part 110E preferably protrudes toward the finger side from an imaginary line connecting the two flat surfaces 161A in a top view. The cylindrical body 110 can be pressed stably by the positioning portion 161. Because the positioning portion 161 comes into contact with the finger, a positional relationship between the finger and the cylindrical body 110 is determined, and thereby the cylindrical body 110 can be pressed stably.

The binding member 160 has a substantially arc shape protruding in a direction spaced from the first opening 110C. A plurality of thin wall portions 162 are formed on the binding member 160. The binding member 160 has a structure that is easily deformable starting from the thin wall portion 162. Bar-shaped members 163 and 164 are provided on a distal end side of the binding member 160. The bar-shaped members 163 and 164 are configured to fit into the cutout portions 154 of the support member 152. The bar-shaped members 163 and 164 of the binding member 160, and the cutout portion 154 of the support member 152 can adjust a fixing position of the binding member 160. The adjustment of the fixing position facilitates the adjustment of a clamping force against the finger and a pressing force of the cylindrical body 110. In addition, the adjustment of the fixing position facilitates adapting to a thickness of the finger that differs depending on people.

By clamping the finger with the two binding members 160, it is possible to easily grasp a region of the finger to be punctured using an instrument attached with a knife such as a lancet. The target region between the two binding members 160 can be easily punctured using the instrument attached with a knife such as a lancet.

A synthetic resin can be applied as a material forming the blood sample guiding instrument 100, and for example, polypropylene or the like can be applied. It is preferable that the cylindrical body 110 and the clamping portion 150 be integrally molded. Thereby, the blood sample guiding instrument 100 is easily manufactured.

The blood sample guiding instrument 100 of the embodiment includes a connecting portion 200, which is connected to an opening of a storing instrument (not shown), on the second opening 110D side of the cylindrical body 110. As shown in FIG. 3, the connecting portion 200 has a structure in which a gap portion 202 that engages with a circumferential edge portion of the opening of the storing instrument is defined. Since the cylindrical body 110 and the opening of the storing instrument are aligned by the connecting portion 200, blood can be reliably transferred to the storing instrument.

The inner circumferential surface 110B of the cylindrical body 110 is preferably water repellent. It is possible to inhibit blood from adhering to the inner circumferential surface 110B, and it is possible to transfer the blood ejected from the finger to the storing instrument. By coating the inner circumferential surface 110B with a water repellent film, the inner circumferential surface 110B can have water repellency. A fluorine-based resin and a silicone-based resin can be applied as the water repellent film. Water repellency can be evaluated by observing a contact angle. In a case where a contact angle is 90° or more, this is evaluated as a “water repellent property.” The contact angle can be measured by an image measuring device or a contact angle measuring device.

<Blood Test Kit>

In addition to the blood sample guiding instrument 100, a blood test kit includes a diluent solution that dilutes the collected blood sample; and a storing instrument that stores the diluted blood sample. The blood test kit is for analyzing a concentration of a target component in the blood sample using a standard component homeostatically present in blood or a standard component that is a standard component contained in the diluent solution but not present in blood.

Furthermore, the blood test kit preferably includes a separating instrument that separates and recovers blood plasma from the diluted blood sample.

[Storing Instrument]

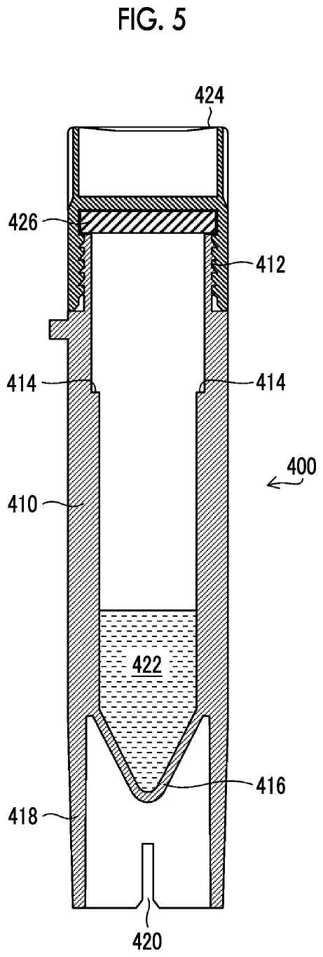

FIG. 5 is cross-sectional view showing an example of a configuration of a storing instrument that stores a diluted blood sample. As shown in FIG. 5, a storing instrument 400 has a cylindrical blood collection container 410 of a transparent material. On an upper end side of the blood collection container 410, a screw portion 412 is formed on the outer surface, and a locking portion 414 is protruded on the inner surface. In addition, a conical bottom portion 416 protruding toward a lower end side is formed at a lower end portion of the blood collection container 410. A cylindrical leg portion 418 is formed around the bottom portion 416. The term “upper” and “lower” mean “upper” and “lower” in a state in which the leg portion 418 is placed on the placement surface.

The leg portion 418 has the same outer diameter as a sample cup (not shown) used at the time of an analytical test of blood, and at positions opposite to the lower end thereof, slit grooves 420 are preferably formed in a vertical direction, respectively. In addition, as shown in FIG. 5, it is preferable that a required amount, for example, 500 mm³ of a diluent solution 422 be stored in the blood collection container 410.

As shown in FIG. 5, it is preferable that an upper end opening of the blood collection container 410 be hermetically sealed with a cap 424 via a packing 426 before using the storing instrument 400.

[Standard Component Homeostatically Present in Blood]

For analysis of a concentration present in plasma of the blood before dilution with respect to a target component after dilution of diluted plasma, in which a dilution factor of blood plasma components is high, it is possible to adopt a method of obtaining from a rate of change in concentration of a substance preliminarily present in the diluent solution. In addition, it is also possible to employ a method for analyzing a concentration of a target component in a blood sample using a standard component homeostatically present in the blood. In a case of analyzing blood components from a smaller amount of blood, a case of employing a method using a standard component homeostatically present in the blood is preferable, because then it is possible to perform measurement with a small measurement error. Accordingly, as the blood test kit of the embodiment of the present invention, the blood test kit for analyzing a concentration of a target component in a blood sample using a standard component homeostatically present in the blood is one of preferred aspects.

“Use” of a standard component means determination of a dilution factor for analyzing a concentration of a target component based on a standard value (homeostatic value in a case of using the standard component homeostatically present in the blood) of the standard component. Accordingly, in a case of analyzing a concentration of a target component in a blood sample using a standard component homeostatically present in blood, it also means analyzing of a concentration of a target component by determining a dilution factor based on a homeostatic value (a standard value) of the standard component homeostatically present in blood.

Examples of standard components homeostatically present in blood include sodium ions, chloride ions, potassium ions, magnesium ions, calcium ions, total proteins, albumins, and the like. Concentrations of these standard components contained in serum and plasma of a blood sample are as follows: a concentration of sodium ion is 134 mmol/L to 146 mmol/L (average value: 142 mmol/L), a concentration of chloride ion is 97 mmol/L to 107 mmol/L (average value: 102 mmol/L), a concentration of potassium ion is 3.2 mmol/L to 4.8 mmol/L (average value: 4.0 mmol/L), a concentration of magnesium ion is 0.75 mmol/L to 1.0 mmol/L (average value: 0.9 mmol/L), a concentration of calcium ion is 4.2 mmol/L to 5.1 mmol/L (average value: 4.65 mmol/L), a concentration of total protein is 6.7 g/100 mL to 8.3 g/100 mL (average value: 7.5 g/100 mL), a concentration of albumin is 4.1 g/100 mL to 5.1 g/100 mL (average value: 4.6 g/100 mL). The embodiment is for making it possible to measure a target component in a case where an amount of blood to be collected is extremely small to ease the pain of a subject, and therefore, in a case of diluting a small amount of blood in a diluent solution, it is necessary to accurately measure a concentration of the “standard component homeostatically present in the blood” present in the diluent solution. As a dilution factor becomes large, a concentration of a component, which is originally present in the blood, in the diluent solution decreases, and depending on dilution factors, measurement errors may be included at the time of measurement of the concentration. Accordingly, it is preferable to measure a standard component present at a high concentration in a small amount of the blood in order to detect the standard component with sufficiently high accuracies in a case where a small amount of blood components is diluted by a large dilution factor. In the present invention, it is preferable to use sodium ions (Nat) or chloride ions (CO which are present at a high concentration among the components homeostatically present in a blood sample. Furthermore, it is most preferable to measure sodium ions which are present in the blood in the largest amount among the above-mentioned standard components homeostatically present in blood. Regarding sodium ions, an average value represents a standard value (a median value within a reference range), and this value is 142 mmol/L accounting for 90 mole % or more of total cations in blood plasma.

[Standard Component not Present in Blood]

One of preferred aspects of the embodiment is a blood test kit for analyzing a concentration of a target component in a blood sample using a standard component not present in the blood. Such a blood test kit may be a kit for using a standard component not present in the blood, together with a standard component homeostatically present in the blood, or may be a kit for using only a standard component not present in the blood without using a standard component homeostatically present in the blood.

In both cases, it is possible to use the standard component not present in blood by adding this standard component into the diluent solution to be described later such that a concentration thereof is a predetermined concentration. As the standard component not present in the blood, it is possible to use a substance which is not contained in the blood sample at all, or is contained therein in an ultra-small amount. As the standard component not present in blood, it is preferable to use substances which do not interfere with the measurement of the target component in the blood sample, substances which do not decompose under the action of biological enzymes in the blood sample, substances which are stable in the diluent solution, substances which do not pass through a blood cell membrane and not contained in the blood cells, substances which are not adsorbed to a storing container of the buffer solution, and substances which can be utilized by a detection system capable of measurement at high accuracy.

As the standard component not present in blood, a substance which is stable even in a case where the substance is stored for a long period of time in a state of being added to the diluent solution, is preferable. Examples of the standard component not present in blood include glycerol triphosphate, Li, Rb, Cs, or Fr as alkali metal, and Sr, Ba, or Ra as alkaline earth metal, and Li and glycerol triphosphate is preferable.

These standard components not present in blood develops color by adding, thereinto, a second reagent at the time of measuring a concentration after blood dilution, and the concentration in the diluted blood can be obtained from a coloring density. For example, regarding measurement of lithium ions added into a diluent solution, a large amount of specimens can be easily measured with a small amount of specimens by using a chelate colorimetric method (a halogenated porphyrin chelating method: perfluoro-5,10,15,20-tetraphenyl-21H,23H-porphyrin) with an automatic biochemistry analyzer. In addition, regarding the measurement of glycerol triphosphate, a large amount of specimens can be easily measured with a small amount of specimens with an automatic biochemistry analyzer by using, for example, concentration measurement of color development of a coloring agent by oxidation condensation, which is described in “Home medical revolution” (clinical examination Vol. 59, p. 397, 2015), which is a known document.

[Diluent Solution]

The blood test kit includes the diluent solution for diluting a collected blood sample. In a case where the blood test kit is for analyzing a concentration of a target component in a blood sample by using a standard component homeostatically present in blood, the diluent solution does not contain a standard component homeostatically present in blood. The phrase “does not contain” in the present specification means that the solution “substantially does not contain” the component. The phrase “substantially does not contain” means that a diluent solution does not contain a homeostatic substance used for obtaining a dilution factor at all, or means a case in which, even in a case where a diluent solution contains a homeostatic substance, an ultra-small amount of concentration is contained to the extent that does not affect measurement of a homeostatic substance in a diluent solution after diluting a blood sample. In a case where sodium ions or chloride ions are used as a standard component homeostatically present in blood, a diluent solution which substantially does not contain sodium ions or chloride ions is used as a diluent solution.

A pH of the blood is generally kept constant from a pH of 7.30 to a pH of about 7.40 in healthy subjects. Therefore, in order to prevent decomposition or denaturation of the target component, the diluent solution is preferably a buffer solution having a buffering action in a pH region within an range of pH 6.5 to pH 8.0, preferably within a range of pH 7.0 to pH 7.5, and more preferably within a range of pH 7.3 to pH 7.4; and the diluent solution is preferably a buffer solution containing a buffer component that suppresses variations in pH.

In the related, as the type of the buffer solution, there are an acetate buffer solution (Na), a phosphate buffer solution (Na), a citrate buffer solution (Na), a borate buffer solution (Na), a tartrate buffer solution (Na), a Tris (tris(hydroxymethyl) aminoethane) buffer solution (CO, a HEPES ([2-[4-(2-hydroxyethyl)-1-piperazinyl]ethanesulfonic acid]) buffer solution, a phosphate buffered saline (Na), and the like. Among these, as a buffer solution around pH 7.0 to pH 8.0, a phosphate buffer solution, a Tris buffer solution, and a HEPES buffer solution are representative. However, the phosphate buffer solution contains a sodium salt of phosphoric acid; the Tris buffer solution has a dissociation pKa of 8.08, and thus is usually used in combination with hydrochloric acid for imparting buffering ability around pH 7.0 to pH 8.0; and a dissociation pKa of sulfonic acid of HEPES is 7.55, but a HEPES mixture of sodium hydroxide and sodium chloride is usually used in order to adjust a buffer solution at constant ionic strength. Therefore, these solutions are useful as a buffer solution having an action of maintaining pH constant, but contain sodium ions or chloride ions which are substances preferably used as an external standard substance in the embodiment, and thus, application thereof to the present invention is not preferable in a case where the blood test kit is for analyzing a concentration of a target component in a blood sample by using a standard component homeostatically present in blood.

In a case where the blood test kit is for analyzing a concentration of a target component in a blood sample by using a standard component homeostatically present in blood, it is preferable that a buffer solution to be used does not contain sodium ions or chloride ions (the meaning of the phrase “does not contain” is as described above). Such a buffer solution is preferably a diluent solution including at least one amino alcohol compound selected from the group consisting of 2-amino-2-methyl-1-propanol (AMP), 2-ethylaminoethanol, N-methyl-D-glucamine, diethanolamine, and triethanolamine, and a buffering agent selected from the group consisting of 2-[4-(2-hydroxyethyl)-1-piperazinyl]ethanesulfonic acid (pKa=7.55) also called HEPES which is a buffering agent having a pKa around 7.4, N-tris(hydroxymethyl)methyl-2-aminoethanesulfonic acid also called TES (pKa=7.50), 3-morpholinopropanesulfonic acid also called MOPS (pKa=7.20), and N,N-bis(2-hydroxyethyl)-2-aminoethanesulfonic acid also called BES (pKa=7.15), which are Good's buffer solutions (Good's buffers). Among these, a combination of 2-amino-2-methyl-1-propanol (AMP) with HEPES, TES, MOPS, or BES is preferable, and a combination of 2-amino-2-methyl-1-propanol (AMP) with HEPES is most preferable. In addition, pKa represents an acid dissociation constant.

For preparing the buffer solution described above, an amino alcohol may be mixed with the Good's buffer solutions at a concentration ratio of 1:2 to 2:1, preferably 1:1.5 to 1.5:1, and more preferably 1:1. A concentration of the buffer solution is not limited, but a concentration of the amino alcohol or Good's buffer solution is 0.1 mmol/L to 1000 mmol/L, preferably 1 mmol/L to 500 mmol/L, and more preferably 10 mmol/L to 100 mmol/L.

A chelating agent, a surfactant, an antibacterial agent, a preservative, a coenzyme, a saccharide, and the like may be contained in the buffer solution in order to keep a target component to be analyzed stable. Examples of chelating agents include ethylenediamine tetraacetic acid (EDTA) salt, citric acid salt, oxalic acid salt, and the like. Examples of surfactants include a cationic surfactant, an anionic surfactant, an amphoteric surfactant, and a nonionic surfactant. Examples of preservatives include sodium azide, antibiotics, and the like. Examples of coenzymes include pyridoxal phosphate, magnesium, zinc, and the like. Examples of saccharides of a red blood cell-stabilizing agent include mannitol, dextrose, oligosaccharide, and the like. Particularly, by adding the antibiotics, it is possible to suppress the growth of bacteria which are partially mixed from the surface of the finger at the time of collecting blood from the finger, suppress decomposition of biological components by bacteria, and stabilize the biological components.

In addition, the buffer solution contains a standard component not present in blood in the blood test kit for analyzing a target component using a standard component not present in blood. It is important that an internal standard substance to be described below is not contained, and a measuring system for blood analysis is not interfered therewith.

From the viewpoint of diluting whole blood, by setting osmotic pressure of the buffer solution equivalent to (285 mOsm/kg (mOsm/kg is an osmotic pressure that 1 kg of water of the solution has, and indicates millimoles of ions)) or higher than that of the blood, it is possible to prevent hemolysis. The osmotic pressure can be adjusted to be isotonic using salts, saccharides, buffering agents, and the like which do not affect measurement of a target component and measurement of a standard component homeostatically present in blood. The osmotic pressure of the buffer solution can be measured by an osmometer.

In a case of testing a specific organ or a specific disease such as liver function, renal function, metabolism, and the like as a blood test, analysis of a plurality of target components to be measured is generally performed at the same time in order to perform a prediction and the like of a state of the organ, a lifestyle habit, and the like by obtaining information of the plurality of target components to be measured which are specific to the organ or the disease. For example, in order to test the state of a liver, generally, a concentration of various types of substances in the blood such as ALT (alanine transaminase), AST (aspartate aminotransferase), γ-GTP (γ-glutamyl transpeptidase), ALP (alkaline phosphatase), total bilirubin, total protein, and albumins is measured. As above, in order to measure the plurality of target components from one blood sample, a certain amount of diluted blood is required in a case of considering a possibility of measuring again. Accordingly, regarding a diluent solution for diluting the collected blood, it is important that a certain amount thereof is secured. However, in consideration of minimizing the invasiveness to a subject, an amount of collected blood is small, and therefore a dilution factor is, for example, 7 times or more, which is a high dilution factor.

(Blood Collection Method and Diluted Blood Sample)

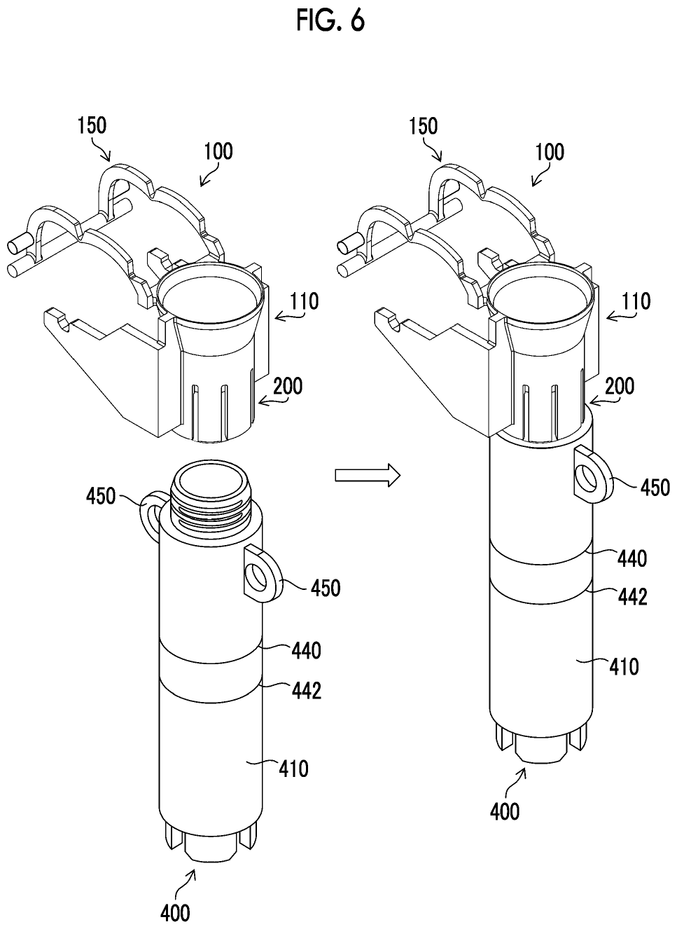

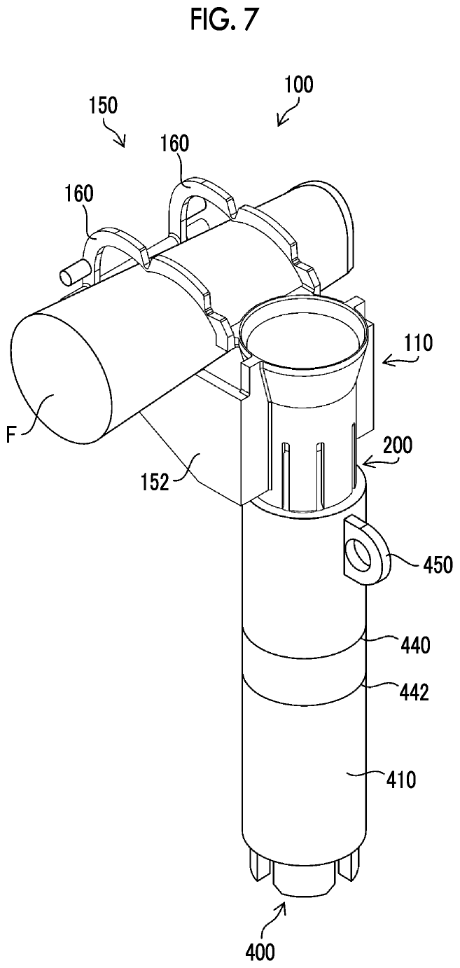

A blood collection method using the above-described blood sample guiding instrument 100 will be described with reference to FIGS. 6 and 7. As shown in FIG. 6, the cap 424 is removed from the blood collection container 410 of the storing instrument 400. The connecting portion 200 of the blood sample guiding instrument 100 and the opening portion of the blood collection container 410 are aligned. The blood collection container 410 preferably has an upper limit scale mark 440 and a lower limit scale mark 442. The upper limit scale mark 440, the lower limit scale mark 442, and a lens effect of the blood collection container 410 make it possible to grasp an amount of blood collected in the blood collection container 410. Furthermore, the amount of blood collected can be more accurately grasped by reducing a diameter of a part of the blood collection container 410 and marking the reduced portion with a scale mark.

The blood collection container 410 preferably includes a strap ring 450 on the outer circumferential surface. By fixing a strap (not shown) to the strap ring 450, the blood collection container 410 can be hung on the neck or the like of a blood collection target subject, and thereby the blood collection container 410 can be prevented from falling. In addition, in order to prevent the blood collection container 410 from falling, it is preferable to provide, for example, a belt (not shown) for fixing to the finger or a fall prevention belt (not shown) for connecting the blood sample guiding instrument 100 and the blood collection container 410.

Next, the blood sample guiding instrument 100 and the blood collection container 410 are connected via the connecting portion 200. The circumferential edge portion of the blood collection container 410 on the opening side is inserted into the gap portion 202 of the connecting portion 200 and engaged therewith.

Next, as shown in FIG. 7, a finger F of the blood collection target subject is clamped by the support member 152 and the binding member 160 which constitute the clamping portion 150, and the cylindrical body 110 is pressed against the finger F. The skin of the finger F between the two binding members 160 is wound using an instrument attached with a knife such as a lancet, and blood is ejected to the outside of the skin. Collection of the blood sample may be performed by a subject himself or by a qualified person such as a doctor.

Blood that has been ejected from the skin is transferred to the blood collection container 410 via the cylindrical body 110 of the blood sample guiding instrument 100. Since the finger F of the blood collection target subject is clamped by the clamping portion 150 and the cylindrical body 110 is pressed against the finger F, the blood is separated from the finger F and transferred to the blood collection container 410. For example, at a time point when it is confirmed that an amount of blood required for a blood test has been transferred to the blood collection container 410 by the upper limit scale mark 440 and the lower limit scale mark 442 which are attached to the blood collection container 410, the blood collection is completed. As a result, the diluted blood sample is stored in the blood collection container 410.

[Separating Instrument]

The blood sample collected by the blood sample guiding instrument 100 may have been in a diluted state for a long time in the storing instrument 400 until analysis is performed thereon. During the time, for example, in a case where red blood cells are hemolyzed, there is a possibility in which test results are affected by elution of substances, enzymes, and the like which are present in the blood cells into the blood plasma or blood serum, or in which an absorption amount of the eluted hemoglobin affects a case of measuring an amount of a target component to be analyzed with light information such as optical absorption of the target component to be analyzed. Therefore, it is preferable that the hemolysis is prevented. For this reason, an aspect in which a separating instrument for separating and recovering blood plasma from a diluted blood sample is contained in a blood test kit is preferable. A preferred example of the separating instrument is a separation membrane. It is possible to use the separation membrane such that blood cells are separated and blood plasma components are recovered by applying pressure to the diluted blood sample, trapping the blood cell components with the separation membrane, and allowing the blood plasma components to pass through the separation membrane. In this case, it is preferable that an anticoagulant is used. In addition, in order to ensure the accuracy of measurement, it is preferable that backflow of the blood plasma passed through the separation membrane to the blood cell side does not occur. Therefore, specifically, the kit can include a backflow prevention means described in JP2003-270239A as a constituent component.

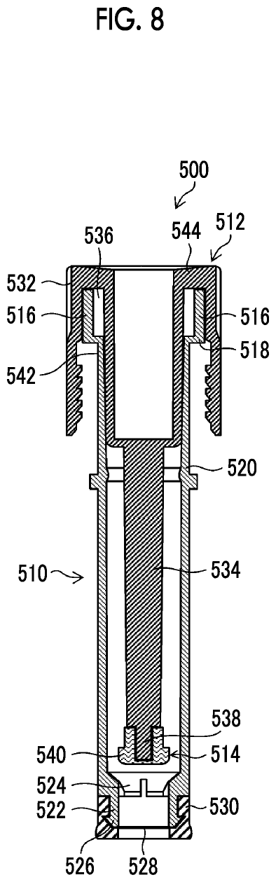

FIG. 8 is a view showing an example of a holding instrument that holds a separating instrument. As shown in FIG. 8, a holding instrument 500 includes a cylinder 510 that can be fitted into the blood collection container 410 of the storing instrument 400 to be inserted thereto, a cap piston 512 attached to the cylinder 510, and a sealing lid 514 functioning as a sealing instrument provided at a lower end of the cap piston 512.

The cylinder 510 is made of a transparent material and has a cylindrical shape. A diameter-increasing portion 516 is formed at an upper end portion 542 of the cylinder 510. The diameter-increasing portion 516 is connected to a main body portion 520 via a thin wall portion 518. A diameter-decreasing portion 522 is formed at a lower end portion of the cylinder 510. A protruded locking portion 524 is formed on an inner surface of the diameter-decreasing portion 522. Furthermore, an outer flange portion 526 is formed at a lower end portion of the diameter-decreasing portion 522. A lower end opening portion of the outer flange portion 526 is covered with a filtration membrane 528 functioning as a separating instrument. The filtration membrane 528 is configured to allow plasma in the blood to pass through and to block passage of blood cells. A cover 530 made of silicone rubber is mounted on an outer circumference of the diameter-decreasing portion 522.

The cap piston 512 is constituted by a substantially cylindrical knob portion 532 and a mandrel portion 534 concentric with the knob portion 532 and extending downward. At an inner upper end portion of the knob portion 532, a cylindrical space 536 into which the diameter-increasing portion 516 of the cylinder 510 is capable of being fitted is formed, and the knob portion is threaded in a lower portion into which a screw can screw. The mandrel portion 534 has a lower end portion 538 formed in a pin shape, and the sealing lid 514 is attachably and detachably provided on the lower end portion 538. The sealing lid 514 is made of silicone rubber. A substantially cylindrical shape in which the lower end portion of the sealing lid 514 is formed in an outer flange shape, and a level difference portion 540 is formed over the outer circumference. The knob portion 532 has a top portion 544, and an inner surface of the top portion 544 and the diameter-increasing portion 516 are in contact with each other.

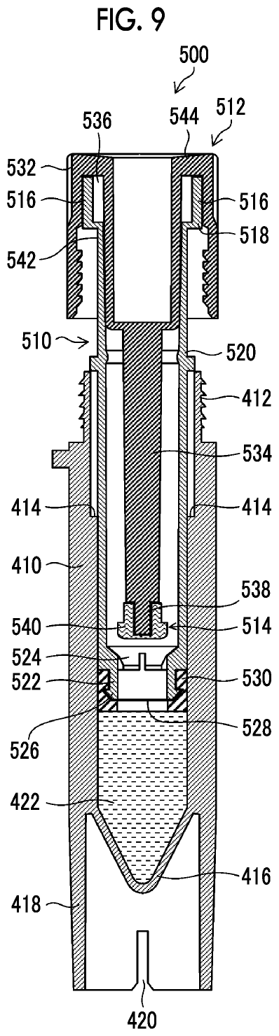

Next, as shown in FIG. 9, in the state of the blood collection container 410 containing the diluted blood sample, the cylinder 510 to which the cap piston 512 is attached is fitted into the blood collection container 410 to be inserted thereto.

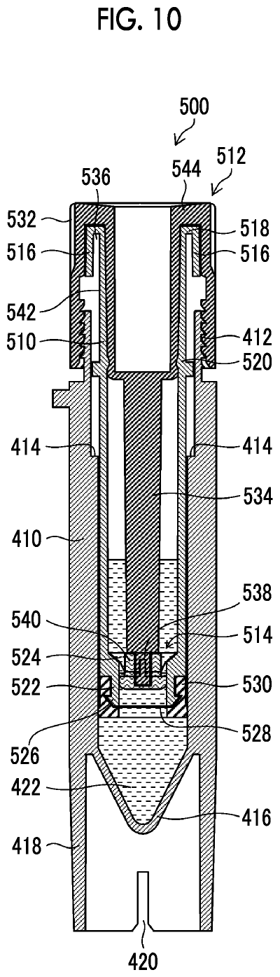

Next, as shown in FIG. 10, the knob portion 532 is screwed into a screw portion 412. Initially, the knob portion 532 and the cylinder 510 rotate. In a case where the locking portion 414 of the blood collection container 410 is engaged with a stopper portion (not shown) formed on an outer circumferential surface of the cylinder 510, the rotation of the cylinder 510 is restrained, and the thin wall portion 518 is broken by twisting. As a result, the cylinder 510 is separated into a main body portion 520 and a diameter-increasing portion 516. Furthermore, in a case where the knob portion 532 is rotated, an upper end portion 542 of the main body portion 520 enters a space 536 inside the diameter-increasing portion 516. Because the cylinder 510 is pressed downward by an inner surface of a top portion 544 of the knob portion 532, the cylinder 510 further descends.

As the cylinder 510 descends, the filtration membrane 528 held by the cylinder 510 moves toward the bottom portion 416 side of the blood collection container 410. In this case, the plasma moves through the filtration membrane 528 to the cylinder 510 side, and the blood cells cannot pass through the filtration membrane 528 and remain on the blood collection container 410 side.

Because an outer diameter of a cover 530 is larger than an outer diameter of the main body portion 520 of the cylinder 510, the cylinder 510 descends in a state of being close contact with the inner surface of the blood collection container 410. Accordingly, in the process of fitting the cylinder 510 into the blood collection container 410 to be inserted thereto, there is no possibility that the diluent solution 422 in the blood collection container 410 leaks to the outside through a gap between the blood collection container 410 and the cylinder 510.

In a case where the knob portion 532 is screwed to the screw portion 412 to the lowermost part, the sealing lid 514 is fitted into the diameter-decreasing portion 522. A flow path between the blood collection container 410 and the cylinder 510 is hermetically sealed by the sealing lid 514. The sealing lid 514 prevents mixing of plasma and blood cells due to backflow.

The blood collection container 410 constitutes a storing instrument in which the diluent solution is stored, and also constitutes a storing instrument for storing a diluted blood sample. In addition, in a state where the cylinder 510 is fitted into the blood collection container 410 to be inserted thereto, thereby separating the plasma and blood cells, the cylinder 510 constitutes a storing instrument for storing recovered plasma. The storing instrument for storing the blood sample corresponds to a combination of the blood collection container 410 and the cylinder 510. That is, the storing instrument for storing a diluted blood sample may be one or a combination of two or more thereof.

The blood test kit is capable of realizing a method that can analyze a target component to be analyzed with high measurement accuracy even in a case where an amount of blood collected is 100 μL or less. The blood test kit is preferably a blood test kit including a manual in which information showing accurate measurement is possible even with a small amount of blood collected, such as 100 μL or less, or showing how much blood sample should be collected by the blood sample guiding instrument 100, and the like.

<Blood Analysis Method>

A blood analysis method using the blood test kit of the embodiment will be described. The blood analysis method includes an aspect which is a medical practice (practice performed by a doctor) for humans and an aspect which is not a medical practice for humans (for example, an aspect in which a person who performs blood collection is a patient himself and an analyzer is a person other than a doctor, an aspect for non-human animals, and the like). The blood analysis method of the embodiment may be performed by the self-blood collection in which a subject to be tested collects blood by himself, or may be performed by the general blood collection in which a qualified person such as a doctor collects blood. As a preferred aspect, a patient pricks the fingertip and the like by himself using an instrument attached with a knife such as a lancet, and then collects blood flowing out of the skin.

A biological specimen which is a target of the present analysis is blood, and the blood is a concept of including serum or blood plasma. Preferably, it is possible to use blood plasma or serum obtained by collecting a small amount of blood from the subject to be tested, diluting the blood with a buffer solution, and then separating blood cells through a filter or by centrifugation. As a component of the blood sample, a blood plasma component separated from a blood sample by a separation means is preferable. The origin of the blood sample is not limited to humans, and may be mammals, birds, fish, and the like which are animals other than humans (non-human animals). Examples of the animals other than humans include horses, cows, pigs, sheep, goats, dogs, cats, mice, bears, pandas, and the like. The origin of a biological specimen is preferably humans.

As a first aspect of the blood analysis method, the analysis of a concentration of a target component is performed by using a standard component homeostatically present in the blood sample. Regarding the standard component homeostatically present in the blood sample, the same explanation in [1] applies thereto.

An occupancy rate of blood plasma components in the blood of a subject to be tested is about 55% in terms of a volume ratio, but varies depending on changes in salt intake and the like of the subject to be tested. Therefore, in the embodiment, a dilution factor of blood plasma is calculated by using a standard value of the standard component which is homeostatically present in the blood plasma, and a concentration of a target component in the blood plasma of a blood sample is analyzed by using the calculated dilution factor. As a method for calculating a dilution factor, it is possible to obtain a dilution factor by calculating a dilution factor (Y/X) of the blood plasma components in a blood sample from a measurement value (concentration X) of an external standard substance (for example, sodium ions and the like) in a diluent solution of the blood plasma, and a known concentration value (concentration Y; in a case of sodium ions, 142 mmol/L) of the external standard substance (for example, sodium ions and the like) contained in the blood plasma of the blood sample. Using this dilution factor, a measurement value (concentration Z) of a target component in a diluent solution of the blood plasma is measured, and by multiplying this measurement value by the dilution factor, it is possible to measure a concentration [Z×(Y/X)] of a target component to be analyzed actually contained in the blood plasma of the blood sample.

A concentration of sodium ions can be measured by, for example, the flame photometric method, the glass-electrode method, the titration method, the ion selective electrode method, the enzyme activity method, and the like. In a particularly preferred aspect, an enzymatic assay utilizing that β-galactosidase is activated by sodium ions, which is that a concentration of sodium ions in a specimen diluted with the diluent solution and galactosidase activity are in a proportional relationship is employed for the measurement of sodium ions.

In addition, in order to confirm whether the blood test kit in which an amount of a standard component derived from members is defined is actually used, or whether a method for diluting blood and recovering blood plasma is normally performed, it is preferable that an additional dilution factor be separately obtained from another standard component in blood plasma so as to check whether a value thereof matches with the dilution factor obtained above. The term “match” means, with respect to two measurement values (a, b), a ratio of a difference thereof to an average value thereof, that is, |a−b|/{(a+b)/2}×100 is 20% or smaller, is preferably 10% or smaller, and is more preferably 5% or smaller. Accordingly, it is possible to verify that analysis of a concentration of a target component in a blood sample has been normally performed. Examples of standard components homeostatically present in blood plasma, which are other than sodium ions and chloride ions, are preferably selected from total proteins or albumins, and are more preferably total proteins. Examples of methods for measuring total proteins include known methods such as a biuret method, an ultraviolet absorption method, a Bradford method, a Lowry method, a bicinchoninic acid (BCA) method, and a fluorescence method. It is possible to appropriately select a method to be used depending on characteristics, sensitivity, a specimen amount, and the like of a measurement specimen.

As a second aspect of the blood analysis method, the analysis of a concentration of a target component is performed by using a standard component not present in blood. In this case, a blood test kit including a diluent solution which contains a standard component not present in blood is used.

As a third aspect of the blood analysis method, the analysis of a concentration of a target component is performed by using a standard component homeostatically present in blood and a standard component not present in blood. Using the two standard components in combination, it is possible to realize the analysis method having higher reliability.

In this case, sodium ions are used as a standard component homeostatically present in blood and lithium ions are used as a standard component not present in blood, and in a case where sodium ions measurement is carried out by the enzyme activity method (to be described later) utilizing that β-galactosidase activity is in a proportional relationship, and lithium ions measurement is carried out by a chelate colorimetric method (to be described later), a dilution factor of the blood sample can be calculated by any one of Formulas 1 to 4.

X=(A+C)/(B+D) Formula 1:

X={(A²+C²)^{1/2}}/{(B²+D²)^{1/2}} Formula 2:

X=a×(B+D)±b Formula 3:

(where a and b are coefficients, and a standard curve represented by Formula 3 is prepared in advance by acquiring data of (B+D) and a dilution factor in advance)

X=A/B′ Formula 4:

(where B′=(A×D)/C)

In the above formulas, A, B, C, D, B′, and X are defined as follows.

A: An absorbance in a case of color development of a buffer solution

B: An amount of change in absorbance after adding blood plasma

C: An absorbance at a median value of 142 mmol/L of blood plasma sodium

D: An absorbance at a concentration of sodium ions after diluting blood plasma

B′: A correction value of an absorbance of a standard component not present in blood of diluted blood plasma obtained, by a dilution factor calculated from the absorbance of the blood plasma sodium

X: A dilution factor of blood plasma

As another calculation method for a case of obtaining a dilution factor, an aspect in which a dilution factor is calculated by Formula 5 using a root-mean-square method, a concentration of a target component to be analyzed in a diluent solution is multiplied by the dilution factor calculated by Formula 5, and a concentration of a target component of components in a blood sample is analyzed, is preferable.

Formula 5:

X=[{(A/B)²+(C/D)²}/2]^{1/2} (1)

A concentration of a target component of components in a blood sample can be calculated from a concentration of a target component in a diluent solution, based on the above-mentioned dilution factor.

The target component to be analyzed is not limited and any substance contained in a biological specimen is a target. Examples thereof include biochemical test items in blood used for clinical diagnosis, markers of various diseases such as tumor markers and hepatitis markers, and the like, and include proteins, sugars, lipids, low molecular weight compounds, and the like. In addition, not only a concentration of a substance is measured, but also an activity of a substance having an activity such as an enzyme is targeted. Measurement of each target component can be carried out by a known method.

In a case of measuring sodium ions, it is possible to use an enzymatic assay by which sodium ions in several μL of a specimen of very low sodium concentration (24 mmol/L or less) diluted with a buffer solution are measured by utilizing that the enzyme activity of the enzyme galactosidase is activated by sodium ions. This method can be applied to a biochemical/automated immunoassay analyzer, and is highly efficient and economical for not requiring another measuring instrument for measurement of sodium ions.

### EXPLANATION OF REFERENCES

100: blood sample guiding instrument
110: cylindrical body
110A: outer circumferential surface
110B: inner circumferential surface
110C: first opening
110D: second opening
110E: contact part
150: clamping portion
152: support member
153: connecting portion
154: cutout portion
160: binding member
161: positioning portion
161A: flat surface
162: thin wall portion
163: bar-shaped member
164: bar-shaped member
200: connecting portion
202: gap portion
400: storing instrument
410: blood collection container
412: screw portion
414: locking portion
416: bottom portion
418: leg portion
420: slit groove
422: diluent solution
424: cap
426: packing
440: upper limit scale mark
442: lower limit scale mark
450: strap ring
500: holding instrument
510: cylinder
512: cap piston
514: sealing lid
516: diameter-increasing portion
518: thin wall portion
520: main body portion
522: diameter-decreasing portion
524: protruded locking portion
526: outer flange portion
528: filtration membrane
530: cover
532: knob portion
534: mandrel portion
536: space
538: lower end portion
540: level difference portion
542: upper end portion
544: top portion

What is claimed is:

1. A blood sample guiding instrument used in a blood test kit, the blood sample guiding instrument comprising:
a cylindrical body in which a first opening and a second opening communicating with the first opening are defined and which comes into contact with a finger; and
a clamping portion that is attached to an outer circumferential surface of the cylindrical body, clamps a finger, and presses the cylindrical body against the finger,
wherein a shape of a part of the cylindrical body which comes into contact with the finger is a curved shape protruding toward a finger side in a top view.

2. The blood sample guiding instrument according to claim 1, wherein the first opening of the cylindrical body is larger than the second opening, and at least a part of an inner circumferential surface of the cylindrical body forms a tapered surface.

3. The blood sample guiding instrument according to claim 1, wherein the clamping portion includes a support member, and at least two binding members that are disposed to be spaced from each other.

4. The blood sample guiding instrument according to claim 3, wherein the binding member adjusts a clamping force for the finger.

5. The blood sample guiding instrument according to claim 3, wherein the binding member is provided at a positioning portion provided on the outer circumferential surface of the cylindrical body.

6. The blood sample guiding instrument according to claim 1, wherein an inner circumferential surface of the cylindrical body has water repellency.

7. The blood sample guiding instrument according to claim 1, further comprising, on a second opening side of the cylindrical body, a connecting portion that is connected to an opening of a storing instrument storing a diluent solution.

8. A blood test kit comprising:
the blood sample guiding instrument according to claim 1 which collects a blood sample;
a diluent solution that dilutes the collected blood sample; and
a storing instrument that stores the diluted blood sample,
wherein a concentration of a target component in the blood sample is analyzed using a standard component homeostatically present in blood or a standard component that is not present in blood but is contained in the diluent solution.

9. The blood test kit according to claim 8, further comprising a separating instrument that separates and recovers blood plasma from the diluted blood sample.
